(19) United States
(12) Patent Application Publication
(10) Pub. No.: US 2025/0205024 A1
(43) Pub. Date: Jun. 26, 2025

(54) DENTURE AND METHODS FOR ADDING AESTHETIC FEATURES TO A DENTURE BASE

(71) Applicant: Harvest Dental Products, LLC, Brea, CA (US)

(72) Inventors: Jorg Erxleben, Brea, CA (US); Sasha Der Avanessian, Cary, NC (US)

(21) Appl. No.: 19/081,338

(22) Filed: Mar. 17, 2025

Related U.S. Application Data
(62) Division of application No. 18/128,455, filed on Mar. 30, 2023.
Application No. 18/128,455 is a continuation of application No. PCT/US22/52332, filed on Dec. 9, 2022.
(60) Provisional application No. 63/361,275, filed on Dec. 13, 2021.

(51) Int. Cl. A61C 13/01; A61K 6/15; A61K 6/62; A61K 6/76; A61K 6/893

(52) U.S. Cl. CPC A61C 13/01; A61K 6/15; A61K 6/62; A61K 6/76; A61K 6/893; A61C 2201/002

(57) ABSTRACT

A composite, three-dimensionally stable facing material for adding aesthetic features to a denture base. The composite may be a flexible, flat strip of material capable of being molded to gingival surfaces of the denture base and retained on the denture base. The strip is capable of being trimmed and then cured to a hardened state. Fibers and/or other characterization may be added for increasing realism of the denture gingival surfaces.

### CROSS REFERENCE TO RELATED APPLICATIONS

This application is a divisional of U.S. patent application Ser. No. 18/128,455 filed Mar. 30, 2023 (pending) which is a continuation of PCT Application No. PCT/US2022/052332 filed Dec. 9, 2022 (expired), which claims the priority of U.S. Provisional Patent Application Ser. No. 63/361,275 filed on Dec. 13, 2021 (expired), the disclosures of which are incorporated herein by reference in their entirety.

### TECHNICAL FIELD

The present invention generally relates to dentures and, more particularly, to manners of adding aesthetic features to a denture to facilitate a more natural appearance.

### BACKGROUND

The aesthetics of a dental prosthesis should harmonize with the soft tissues that surround the dentition, especially in the anterior region. This desire is primarily so that the prosthetic is as undetectable as possible during use by the wearer. Materials such as polymethylmethacrylate have proven to be very successful base materials for dentures. Other materials, such as conventional acrylic denture resins, offer more limited aesthetics because they do not have the natural appearance of healthy gingiva. A healthy looking gingiva is the result of the combination of a bone structure covered by a somewhat transparent, veined pinkish gingival tissue. And while the aesthetics of the tooth surfaces of a denture are important, the gingival surfaces are often more easily detected as being prosthetic. One method of making these surfaces look more realistic is to utilize shaded composite pastes, liquids and fibers to build up and model the visible gingival surfaces. Composite pastes are used primarily to add texture and dimension to the gingival surfaces of the prosthetic, while liquid modifiers are used to vary the coloration to look more like natural gingival tissues. Short fibers and colorants are also added to represent small gingival veins just below the surface. The final surface is then typically ground and polished. While the results of this buildup and bonding process can provide very good results, the technique of manually sculpting and brushing these various layers of materials, which includes multiple curing/bonding steps, is quite time-consuming and highly dependent on the artistic abilities of the technician.

Although various products are available to a dental technician to perform a layering buildup on gingival surfaces of a restoration to provide aesthetics more similar to natural gingival tissues, industry would find advantage in new methods and materials which simplify this process and provide more consistent results. It would therefore be desirable to provide composite products, systems and manners for achieving a highly authentic, realistic gingival surface for a denture that are less time consuming and/or less dependent on specialized technician artistry or skills.

### SUMMARY

Generally, a chemically bondable composite facing material is provided for adding texture and color to the gingival area of a denture base structure. This may take the form of a three-dimensionally stable, malleable facing material applied to a dentine/bone-like substructure of the denture. The facing material may include anatomical features such as replicas of gingival veins formed beneath, within and/or on an outer surface thereof, as well as various shades of coloration. These replica veins may, for example, include fibers incorporated on the outside surface of the facing material, within the facing material or beneath the facing material or in any combinations of these locations. Any other features may be added to make the denture more life-like. The facing material may be easily cut, bonded to and hand worked on the gingival surface of the denture or restoration base, then trimmed and finished with appropriate features such as the gingival line and interdental papilla. Liquid modifiers (e.g., stains) may be used for enhanced characterization resembling the natural gingiva. “Characterization” is defined as modifications to the form and/or coloring/shading to achieve a more life-like appearance. Finally, the facing material may be cured, such as by using visible light, to a final hardness and then polished as necessary. Any other manner of curing the facing material may be used depending on the formulation of the material (e.g., the initiator used). As non-limiting examples, the facing material may be self-curing, heat-curing, moisture-curing, electron beam curing or any combination of curing types. All of this is completed without an onerous layering process of pastes or similar materials, or extensive hand and tool forming of gingival features.

In an illustrative embodiment, the invention provides a composite flat facing material for adding aesthetic features to a denture base. The composite includes a flexible, flat facing material capable of being molded to gingival surfaces of a denture base and retained on the denture base, the facing material further being capable of being trimmed and then cured to a hardened state. As examples, the facing material may be formed as a flat strip of material, a flat sheet of material or rolled forms of flat material such as these.

The facing material may be formed at least in part from a polymeric material. A specific example for the material is provided below in the Example section. As more general examples, the following material compositions may be used:
Oligo/Poly-Urethane Di-(Tri) Methacrylates Viscosity 4,000-20.000 cp (CAS #72869-86-4)
Oligo/Poly-Alkyleneglycol Di-(Tri) Methacrylates Viscosity 4,000-20.000 cp (CAS #25852-47-5)
Oligo/Poly-Epoxy Di-(Tri) (Meth-)acrylates Viscosity 4,000-20.000 cp (CAS #71281-65-7)
with a wide molecular weight/viscosity range.

The facing material is chemically bonded to the denture base. An adhesive may be provided for chemically bonding the facing material to the gingival surfaces of the denture base. The adhesive may be applied to the facing material prior to bonding the facing material to the gingival surfaces of the denture base and/or the adhesive may be separate from the facing material. Alternatively, or in addition, the denture base material and the facing material may be designed to facilitate a chemical bond therebetween. The facing material may be light-curable and may be at least partially translucent. The facing material may be packaged as a flat strip (e.g., a flat rectangular shape), a flat sheet of any shape or in a rolled condition similar to a roll of tape. The packaging should maintain the facing material protected from light and mechanical damage. Other packaging precautions may be taken based on the needs of the material. For example, if the material is moisture- curable, the packaging should impart moisture-proof conditions. The flat facing material may be textured on its surface(s) during manufacture, if desired.

In another illustrative aspect, a denture is provided and includes a denture base, a flexible, flat facing material molded to gingival surfaces of the denture base and retained on the denture base to provide the look of natural gingival surfaces. The facing material may be formed at least in part from a polymeric material. The denture may further include an adhesive located between the denture base and the facing material for bonding the facing material to the gingival surfaces of the denture base. The adhesive may be applied to the facing material prior to bonding the facing material to the gingival surfaces of the denture base, and/or the adhesive may be separate from the facing material. Other manners of chemical bonding may be used too. The facing material may be light-curable and may be at least partially translucent. The denture may further include fibers for replicating a vein structure of gingival tissue and may include liquid modifiers to provide coloring to the facing material.

In another illustrative aspect, the invention further provides a method of adding aesthetic features to a denture base. The method includes applying a flexible, flat facing material to gingival surfaces of the denture base, trimming the facing material to conform to the gingival surfaces of the denture base and curing the facing material into a hardened condition. The facing material may be formed at least in part from a polymeric material. The method may further include using an adhesive to bond the facing material to the gingival surfaces of the denture base. For example, the method may further include applying an adhesive to the gingival surfaces of the denture base to bond the facing material to the gingival surfaces of the denture base. Alternatively, or in addition, the method may include using an adhesive applied to the facing material to bond the facing material to the gingival surfaces of the denture base. The step of curing the facing material further may include using light to cure the strip. Any other alternative curing methods such as, but not limited to, electron beam, self-curing, heat induced polymerization or others are applicable. Fibers may be used to replicate a vein structure of gingival tissue and liquid modifiers may be used to provide characterization to the facing material.

Additional features and advantages of the inventive aspects will become more apparent upon review of the following detailed description taken together with accompanying drawings of the illustrative and exemplary embodiments.

### BRIEF DESCRIPTION OF DRAWINGS

FIG. 1 is a top perspective view of a flexible, malleable aesthetic strip of facing material about to be applied to a denture base.

FIG. 1A is a side elevational view of the denture base shown in FIG. 1, with the flexible, malleable strip of facing material schematically shown in dash-dot lines just prior to being applied and molded to the denture base.

FIG. 2 is a top perspective view of the flexible, malleable aesthetic strip of facing material being applied to the denture base.

FIG. 3 is a top perspective view of the flexible, malleable aesthetic strip of facing material being molded by hand to the denture base.

FIG. 4 is a top perspective view of the flexible, malleable aesthetic strip of facing material after being molded and adhered to the denture base, and undergoing a trimming process.

FIG. 4A is a side elevational view of the flexible, malleable aesthetic strip of facing material after being molded and adhered to the denture base, and undergoing a trimming process as illustrated in FIG. 4.

FIG. 5 is a top perspective view of the denture base and the molded, adhered and trimmed strip of facing material being cured into a hardened condition.

FIG. 6 is a top perspective view of the denture base and the molded, adhered, trimmed and cured strip of facing material being polished.

FIG. 6A is a side elevational view of the denture shown in FIG. 6 being polished.

FIG. 7 is a top elevational view of the denture shown in FIGS. 6 and 6A being colored, such as with a stain applied with a brush.

FIG. 8 is a perspective view of a strip of facing material constructed in accordance with an illustrative embodiment in a flat, rectangular form.

FIG. 9 is a perspective view of a strip of facing material constructed in accordance with an illustrative embodiment in a roll form.

FIG. 10 is an elevation view of the facing material in a flat, sheet form.

### DETAILED DESCRIPTION

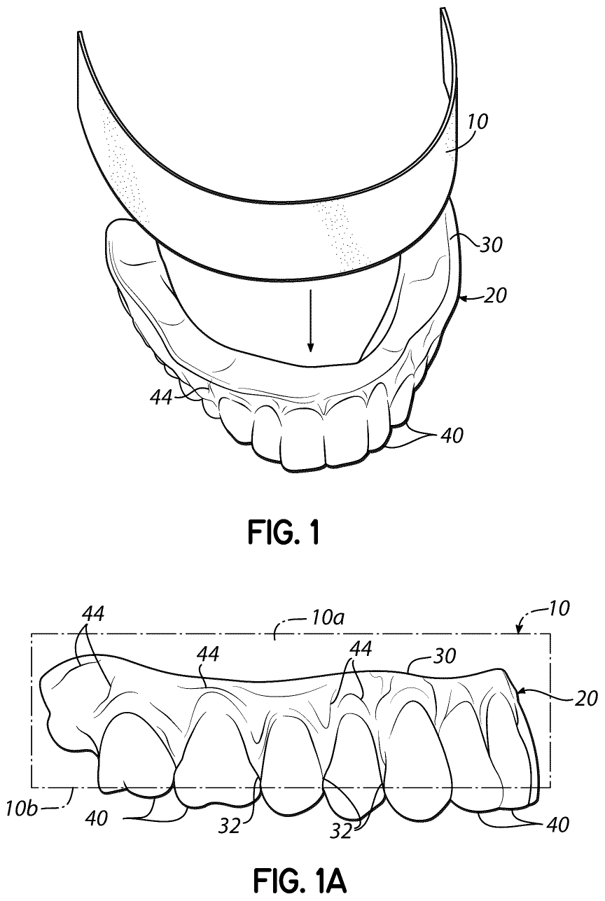

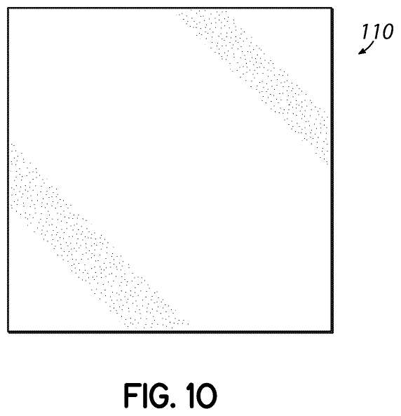

An illustrative embodiment of the invention relates to a three-dimensionally stable, biocompatible dental facing material that has sufficient strength to maintain its shape as a three-dimensionally stable, flat material during transportation and storage, yet has sufficient malleability to be subsequently formed or molded, such as by hand, into a final shape on the gingival surfaces of the denture base and then hardened and polished. This material may be used as a veneer to improve the aesthetics of the gingival portion of a removable dental prosthetic or denture. The facing material is preferably comprised of a curable composite formed at least partially of a polymeric material. Any shape other than the relatively narrow strip shape shown in FIGS. 1-9 may be used including, but not limited to, the sheet form shown in FIG. 10. The format of the facing material may be obtained in any desired manner including, e.g., extrusion, molding and/or rolling and cutting. The material may be cured by the user with visible light or in any other manner depending on the composition of the facing material. The facing material is sufficiently strong, prior to curing, that the three-dimensionally stable pre-formed flat shape and its features, such as malleability, workability, color retention and curability, are retained prior to use, including during transportation (e.g., shipping) and storage. This means that the facing material remains dimensionally stable while contained in its packaging, for sufficient time (e.g., one year at about 75° F. or room temperature) as well as during temporary spikes in temperature up to about 110° F. and down to about 0° F. The facing material will be sufficiently malleable prior to curing for purposes of allowing final features to be formed, such as with easy manual manipulation or working by a dental technician. Preferably, the facing material is easily formed or worked by hand, prior to curing, under a moderate force, using fingers and/or typical dental hand tools.

The shape and size of the facing material, with or without the need for cutting to size by the technician, will facilitate its use as a gingival veneer, being generally rectangular in shape, and the facing material thickness will range from about 0.5 mm to about 2 mm. The width of a useful strip of this material may range from about 10 mm to about 25 mm. Length of the strip may be sufficient to cover the gingival surfaces of a full denture, e.g., up to about 100 mm. Sheets of facing material may alternatively be formed and then cut out to individual shapes and sizes for use. Such sheets may be sized in any useful dimension(s). The length may also be considered unlimited, such as where a strip of the facing material is provided on a roll like a roll of tape. In this form, the technician will cut desired lengths of the strip of material from the roll.

The facing material may be pre-formed during manufacture with coloring in various shades of pink to red, which are then selected for the shade most similar to natural gingiva desired for a particular patient. The coloring may be partially translucent, translucent and/or opaque. The facing material is then cut and trimmed to the approximate length and width of the area to be covered. The area on the denture base to receive the facing material is cleaned, then the facing material is applied onto the desired location such as with the use of a bonding agent. Additional trimming and forming is completed as needed (such as at the gingival line and interdental papilla) to mimic the natural gingiva. Liquid modifiers may be used to enhance the life-like characterization of the acing material, and once the technician is pleased with the aesthetics of the gingival portion of the denture, the facing material is cured to full hardness. The prosthetic or denture may then be polished as needed and delivered to verify fit and aesthetics for the patient.

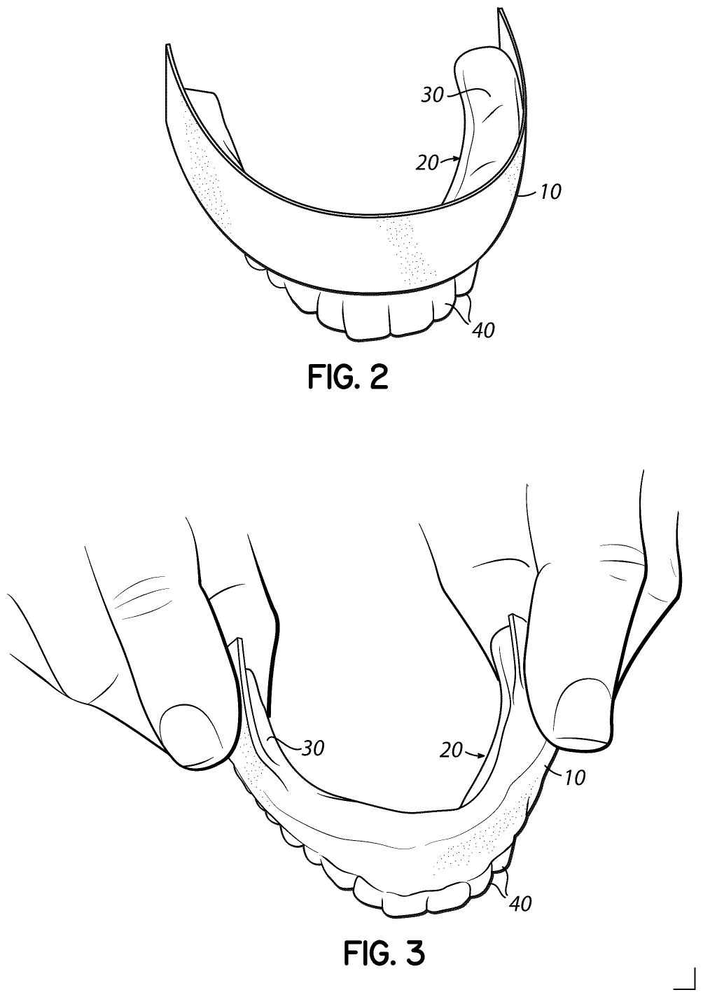

Referring to FIGS. 1 and 1A, a strip of facing material 10 is positioned relative to a denture base 20 such that an upper portion 10a of the strip extends above the upper gingival portions 30 of the denture base 20 and a lower edge 10b of the strip 10 extends below the lowermost points 32 of the gingiva between the prosthetic teeth 40. A bonding agent (not shown) may be first applied to the gingival surfaces 42 wherever the strip of material 10 will be in contact. Alternatively, or in addition, a bonding agent (not shown) may be applied by the technician, or pre-applied or incorporated during the manufacturing process of the strip 10, to the strip of material 10. The bonding agent may be an adhesive, such as a ready-to-use adhesive or an adhesive component. Veins may be similated by adding suitable fibers 44. These fibers 44 may be adhered to the denture base 20, as shown, and/or incorporated into or onto the strip 10 as desired for enhancing realism. As further shown in FIGS. 2 and 3, the strip of material 10 is hand molded or worked by pressing the strip 10 against the prosthetic gingival surfaces adjacent to the teeth 40 and upper gingival portions 30 as particularly shown in FIG. 3.

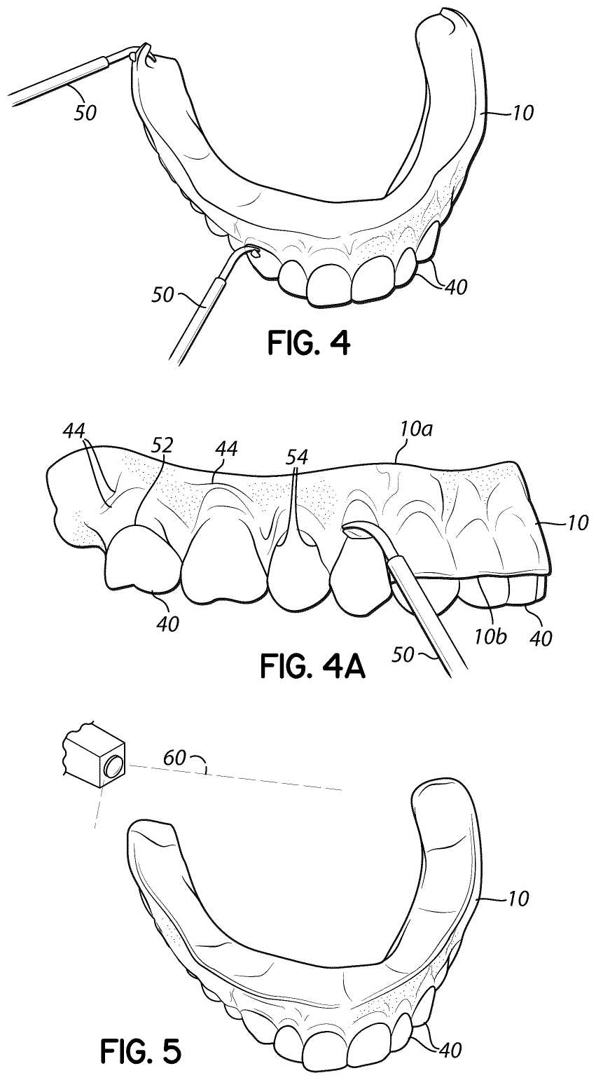

As shown in FIGS. 4 and 4A, dental hand tools 50 are used to appropriately trim the strip of material 10 to create realistic gingival lines 52 and papilla lines 54 adjacent the prosthetic teeth 40.

As further shown schematically in FIG. 5, light 60 is used to cure the hand worked or molded and trimmed strip of material 10 after the technician is satisfied with the aesthetics, such that the strip of material 10 is fully hardened. The light 60 may be of any wavelength necessary to cure the light-curable material formulation used for the strip of material 10. Preferably, the light 60 is in the visible portion of the spectrum. As discussed, any other curing method may be used depending on the chemical formulation of the facing material.

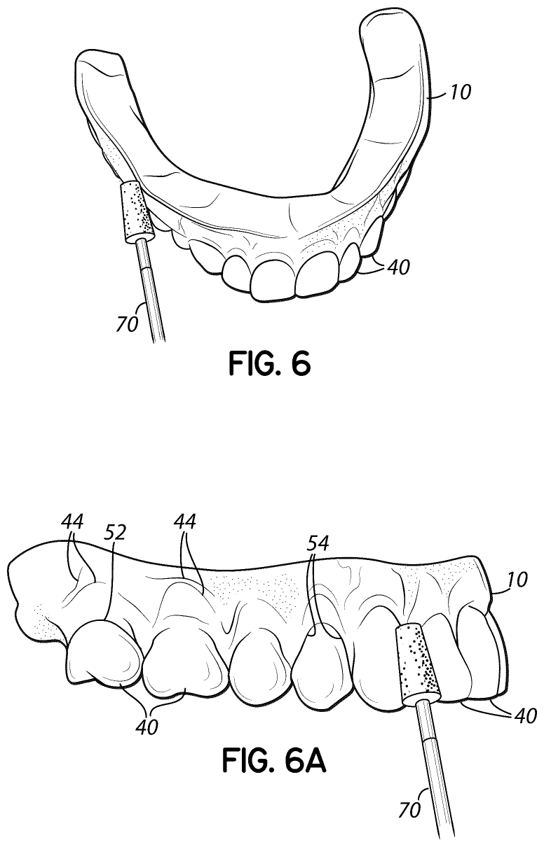

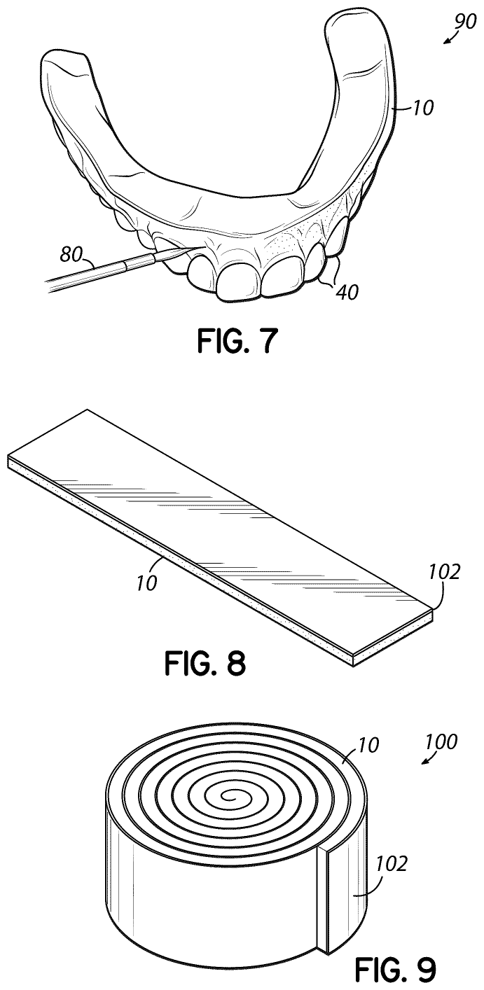

As further shown in FIGS. 6 and 6A, the fully hardened material 10 is then polished by one or more tools 70 as needed or desired by the technician to achieve the desired look. As further shown in FIG. 7, characterization may be added by the technician, such as by using an application brush 80 and/or other tool brushing stain or otherwise applying color to the visible surfaces of the prosthetic gingiva comprised of the strip 10. Other manners of adding life-like characterization may be used depending on the desires of the technician. The denture 90 is then complete and may be fitted to the patient.

FIGS. 8 and 9 illustrate two possible forms for the strip of material, including a rectangular strip 10 shown in FIG. 8 and another form of the strip of material 10 that has been placed into a rolled condition 100, similar to a roll of tape, shown in FIG. 9. Each of these forms may include a release film or paper 102 which is peeled off to expose an adhesive side of the strip 10.

FIG. 10 illustrates a sheet 110 of the facing material. Sheet 110 may have any desired shape and/or dimensions from which smaller and more narrow strips of the facing material may be cut or otherwise removed or separated for use. As one of many possible examples, the sheet may be 200 mm(l)×200 mm(w)×2.5 mm(t). Of course, any other size may be used instead.

### EXAMPLE

An illustrative and preferred formulation of the facing material is comprised of:
17.06% Urethane Methacrylate Viscosity 8,000 cp (CAS #72869-86-4)
5.60% TMPTMA (Trimethylolpropane Trimethacrylate (CAS #3292-92-4)
22.10% PEG200DMA (CAS #25852-47-5)
55.10% Surface Treated Pyrogenic Silica (200 m²/g) (CAS #112945-52-5)
0.06% 2,4,6-trimethylbenzoyldiphenyl phosphine oxide (CAS #1628881-26-7)
0.03% Camphorquinone (CAS #10373-78-1)
0.03% EDMAB (CAS #10287-53-3)
0.02% 3,5-Di-tert-4-butylhydroxytoluene (CAS #128-37-0)

The formulation presents a Flexural Strength of 99 MPa and a Flexural Modulus of 4,400 MPa for the strip of material.

One illustrative and preferred strip size is: 70 mm (length)×10 mm (width)×2.5 mm (thickness).

There may be various translucent, semi-translucent and opaque shades of the facing material. Any of these may include different levels of veining provided, such as by using fibers (which may or may not be formed into a fibrous material or mat) incorporated within the facing material and/or applied on the facing material. The coloring or shading may be various shades of pink and/or red.

A modifier liquid stain may be formed using the same formulation as above, but with 25% less Pyrogenic Silica in Red, Brown, Black, Blue, Yellow and Dark Pink. Special attention should be given to the possibility to characterize underneath the “protecting” overlayed composite facing material, which provides a protection to the aesthetics of the facing material.

An illustrative and preferred bonding formulation applied to the denture base before applying the strip is comprised of:
50.0% Acetone (CAS #67-64-1)
25.0% Urethane Methacrylate Viscosity 8,000 cp (CAS #72869-86-4)
25.0% TMPTMA (Trimethylolpropane Trimethacrylate (CAS #3292-92-4)

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. A denture comprising:
a denture base;
a flexible and the three-dimensionally stable facing material hand molded to gingival surfaces of the denture base and retained on the denture base to provide the look of natural gingival surfaces.

2. The denture of claim 1, wherein the facing material is formed at least in part from a polymeric material.

3. The denture of claim 1, further comprising an adhesive located between the denture base and the facing material for bonding the facing material to the gingival surfaces of the denture base.

4. The denture of claim 3, wherein the adhesive is applied to or incorporated in the facing material prior to bonding the facing material to the gingival surfaces of the denture base.

5. The denture of claim 3, wherein the adhesive is separate from the facing material.

6. The denture of claim 1, wherein the facing material is at least one of light curable, electron beam curable, moisture curable, heat curable or self-curing.

7. The denture of claim 1, wherein the facing material is at least partially translucent.

8. The denture of claim 1, further comprising fibers for replicating a vein structure of gingival tissue.

9. The denture of claim 1, further comprising liquid modifiers to provide characterization to the facing material-modified denture.

10. A method of adding aesthetic features to a denture base, the method comprising:
applying a flexible and three-dimensionally stable facing material to gingival surfaces of the denture base;
hand molding the facing material to conform to the gingival surfaces of the denture base;
trimming the facing material to conform to the gingival surfaces of the denture base; and
curing the facing material into a hardened condition.

11. The method of claim 10, wherein the facing material is formed at least in part from a polymeric material.

12. The method of claim 10, further comprising using an adhesive to bond the facing material to the gingival surfaces of the denture base.

13. The method of claim 10, further comprising using an adhesive applied to or incorporated in the facing material to bond the facing material to the gingival surfaces of the denture base.

14. The method of claim 10, further comprising applying an adhesive to the gingival surfaces of the denture base to bond the facing material to the gingival surfaces of the denture base.

15. The method of claim 10, wherein the step of curing the facing material further comprising curing the facing material using at least one of light, electron beam, moisture, heat or self-curing.

16. The method of claim 10, further comprising using fibers to replicate a vein structure of gingival tissue.

17. The method of claim 10, further comprising using liquid modifiers to provide characterization to the facing material-modified denture.
